(19) United States
(12) Patent Application Publication
(10) Pub. No.: US 2012/0167885 A1
(43) Pub. Date: Jul. 5, 2012

(54) Systems And Methods For Ventilation To Obtain A Predetermined Patient Effort

(75) Inventors: Milenko Masic, San Diego, CA (US); Peter Doyle, Vista, CA (US)

(73) Assignee: Nellcor Puritan Bennett LLC, Boulder, CO (US)

(21) Appl. No.: 12/980,583

(22) Filed: Dec. 29, 2010

(51) Int. Cl. A61M 16/00

(52) U.S. Cl. 128/204.23

(57) ABSTRACT

Various embodiments of the present disclosure provide systems, methods and devices for respiratory support. As one example, a ventilation system is disclosed that includes a computer readable medium including instructions executable by a processor to receive a measured pressure value and a net flow value. A patient effort value is calculated based on a relationship between patient effort, the measured pressure value and the net flow value. The instructions are further executable to calculate a gas delivery metric based on the computed patient effort to obtain a predetermined patient effort. Gas is then caused to be delivered consistent with the gas delivery metric.

### INTRODUCTION

Medical ventilator systems have been long used to provide supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient.

Modern ventilators are dynamic systems whose dynamic behavior and outputs, such as pressures and flows delivered to the patient, are driven by input signals, such as gas flows. Proper operation of such ventilators relies on some understanding of a variety of respiratory parameters including the resistance of the patient airways and the compliance of the lung. These parameters may vary significantly from one ventilation system to another and from one patient to another. In many cases, proper operation of a ventilation system is limited by the accuracy at which such parameters are defined or estimated.

Historically, identifying respiratory parameters posed a challenge in the case of the ventilation system driven by unknown input signals, such as in ventilation systems involving actively breathing patients and leaks. For example, various approaches for estimating patient breathing effort are inaccurate, and as such dynamic methods relying on an estimated patient effort are often inadequate.

### SUMMARY

The present disclosure is related to ventilators, and more particularly to systems and methods for controlling the delivery of gas based at least in part on a computed patient effort to obtain a predetermined patient effort.

In part, this disclosure describes a ventilation system, including: a processor communicably coupled to a computer readable medium, wherein the computer readable medium includes instructions executable by the processor to: receive a measured pressure value, receive a net flow value, calculate a patient effort value based on a relationship between patient effort, the measured pressure value and the net flow value; update an interim value based at least in part on the patient effort value; calculate a computed patient effort based at least in part on the interim value; calculate a gas delivery metric to obtain a predetermined patient effort, wherein the gas delivery metric is based on the computed patient effort value; and cause a gas to be delivered consistent with the gas delivery metric.

Yet another aspect of this disclosure describes a method for respiratory support. The method includes:
a) measuring a pressure and providing a measured pressure;
b) measuring an inlet flow and an outlet flow, and providing a measured net flow;
c) using a relationship between a first value related to the measured pressure, a second value related to the measured net flow and a third value related to patient effort to calculate a prediction of patient effort from the measured pressure and measured net flow;
d) updating an interim value based at least in part on the prediction of patient effort;
e) calculating a computed patient effort based at least in part on the interim value; and
f) supplying a gas based on the computed patient effort to obtain a predetermined patient effort. In yet another aspect, the disclosure describes a ventilation system that includes: a gas inlet; a gas outlet; a tube coupling the gas inlet and the gas outlet; a pressure sensor, wherein the pressure sensor is operable to provide a measured pressure value indicating a pressure in the tube; a first flow sensor, wherein the first flow sensor is operable to provide an inlet flow value indicating a flow associated with the gas inlet; a second flow sensor, wherein the second flow sensor is operable to provide an outlet flow value indicating a flow associated with the gas outlet; and a processor communicably coupled to a computer readable medium, wherein the computer readable medium includes instructions executable by the processor to: receive a measured pressure value, receive a net flow value, calculate a patient effort value based on a relationship between patient effort, the measured pressure value and the net flow value, update an interim value based at least in part on the patient effort value, calculate a computed patient effort based at least in part on the interim value, calculate a gas delivery metric based on the computed patient effort to obtain a predetermined patient effort, determine an inhalation phase based on the computed patient effort, and cause a gas to be delivered consistent with the gas delivery metric.

In an additional aspect, the disclosure describes a ventilation system that includes: means for measuring a pressure and providing a measured pressure; means for measuring an inlet flow and an outlet flow, and providing a measured net flow; means for using a relationship between a first value related to the measured pressure, a second value related to the measured net flow and a third value related to patient effort to calculate a prediction of patient effort from the measured pressure and measured net flow; means for updating an interim value based at least in part on the prediction of patient effort; means for calculating a computed patient effort based at least in part on the interim value; and means for supplying a gas based on the computed patient effort to maintain a predetermined patient effort.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments systems and methods described below and are not meant to limit the scope of the invention in any manner, which scope shall be based on the claims appended hereto.

FIG. 1 depicts a ventilation system including, among other things, an adaptive calculation module capable of providing adaptively estimated respiratory parameters and patient effort in accordance with various embodiments of the present invention.

FIG. 2 shows a patient ventilator system and associated parameterized model that may be used for determining patient effort in accordance with some embodiments of the present invention.

FIG. 3 provides a graphical example of patient effort correlated to other signals that is achievable through implementation of a particular embodiment of the present invention.

FIG. 4 is a flow diagram depicting a method in accordance with some embodiments of the present invention for determining patient effort; for determining patient effort in accordance with various embodiments of the present invention.

FIG. 6 is a flow diagram depicting a method in accordance with some embodiments of the present invention for triggering a ventilation cycle.

FIG. 7 is a timing diagram showing triggering a ventilation cycle based upon an estimated patient effort signal in accordance with various embodiments of the present invention.

FIG. 8 are timing diagrams comparing a process triggering off of a pressure sensor verses triggering off of an estimated patient effort signal in accordance with one or more embodiments of the present invention.

FIG. 9 is a flow diagram showing a method for providing ventilation in proportion to patient effort in accordance with various embodiments of the present invention.

FIG. 10 illustrates a group of timing diagrams that graphically depict providing ventilation in proportion to patient effort in accordance with one or more embodiments of the present invention.

FIG. 11 shows an exemplary graphical interface showing the display of patient effort corresponding to an actively breathing patient in accordance with some embodiments of the present invention.

FIG. 12 shows an exemplary graphical interface showing the display of a respiratory parameter corresponding to an actively breathing patient in accordance with some embodiments of the present invention.

FIG. 13 is a flow diagram showing a method for providing ventilation to obtain a predetermined patient effort in accordance with various embodiments of the present invention.

### DETAILED DESCRIPTION

The present disclosure is related to ventilators, and more particularly to systems and methods for controlling the delivery of gas based on a patient's effort to breathe.

It is desirable to synchronize the onset and end of a ventilation cycle to effort a patient may be making to breathe on their own (i.e., patient effort). For example, it is desirable to have an accurate ventilator trigger, whereby the ventilator initiates a breath as soon as the patient attempts to inhale. Some ventilators use a pressure trigger which senses a change in ventilation circuit pressure caused by the patient attempting to inhale, while other ventilators use a flow trigger which senses a change in flow caused by the patient attempting to inhale. In either case, delays between the patient's effort and the ventilator response can occur due to a variety of reasons. For example, a leak in the ventilation circuit may allow air to enter the circuit when the patient inhales. Since the entirety of the patient breath is not measured by a ventilator flow sensor, and the ventilator may be monitoring a change in flow to detect an inhalation (flow trigger), the ventilator may be delayed in initiating the breath. Some embodiments of the present invention facilitate improved synchronization through providing a reasonably accurate estimate of patient effort that may be used either alone or in relation to other signals to trigger the onset and end of a ventilation cycle. In one or more embodiments of the present invention, the estimated patient effort may be additionally used in relation to controlling proportional ventilation of a patient. Such proportional ventilation operates to deliver a gas to a patient in proportion to the patient's effort to receive such gas. In various embodiments of the present invention, the estimated patient effort and/or respiratory parameters may be used to drive a graphical display that may be used by a clinician for patient monitoring and/or diagnostic purposes.

Various embodiments of the present disclosure provide systems and methods for estimating of one or more respiratory parameters and at least one un-measured input signal driving a ventilation system with a reasonable degree of accuracy. In some embodiments, at least one un-measured input signal may be derived from measured input signals, such as measured pressure and measured flow, and used to estimate the respiratory parameters. The un-measured input signal may be, but is not limited to, patient effort and/or a derivative of patient effort, a ventilation system gas leak (i.e., a leak occurring in the tubing or patient interface connecting a ventilator to a patient), a patient gas leak (e.g., a leak in the patient's lung), and/or flow and pressure sensing errors. The respiratory parameters may include, but are not limited to, lung compliance (C_{I}), patient resistance (R_{P}), and tubing compliance (C_{T}). In some cases, estimation of both respiratory parameters and the un-measured input signal(s) is simultaneous. In some embodiments, the un-measured input signal has a strong correlation to patient effort, and therefore can be used as a surrogate for patient effort in subsequent ventilator actions. In other embodiments, methods of the present invention allow the respiratory parameters to be continuously provided. In this manner, patient effort may be determined, as well as respiratory or ventilation system parameters such as lung compliance, patient resistance, leak, etc.

In some embodiments of the present invention, a relationship between measurable pressure, measurable flow and an unknown patient effort is exploited to provide a continuous estimate of patient effort along with a variety of respiratory parameters. In particular instances, the relationship is defined as a transfer function relating, inter alia, measured pressure, measured flow and patient effort. In such cases, the transfer function may be reduced using linear regression techniques to yield one or more interim values that may in turn be used to estimate patient effort. In an embodiment, ongoing inputs of measured pressure and measured flow are plugged into the transfer function to estimate patient effort and, as needed, one or more respiratory parameters. In another embodiment, the estimate of patient effort may be used recursively to derive a more accurate estimate of patient effort during succeeding calculation periods. Thus, through use of recursion, the accuracy of an estimated patient effort value may be continuously improved.

In some cases, the measured flow is a net flow value that combines a net flow of gas out of the system with a net flow of gas into the system. In one particular case, the net flow of gas into the system includes a flow of Oxygen combined with a flow of Air into the system. Such flows are reasonably easy to measure, and are not subject to the inaccuracies that often attend the measurement of gas flow near the lung.

In some cases, a patient effort signal or some proxy thereof calculated as described above may be used to trigger a ventilation cycle. Use of such signals can allow a ventilation system to more accurately synchronize mechanical ventilation with the efforts being made by a patient to breathe on their own.

Of note, the respiratory parameters and the derivative of patient effort may be inputs to the same model, and may be calculated using interdependent equations derived from that same model. As the values calculated from some of the interdependent equations are used as inputs to other interdependent equations, they may be generically referred to as interim values. As used herein, the phrase “interim value” is used in its broadest sense to mean a value derived from one equation that is used as an input to another equation. It will be noted based on reading this disclosure that a variety of interim values may be utilized in relation to the various embodiments of the present invention.

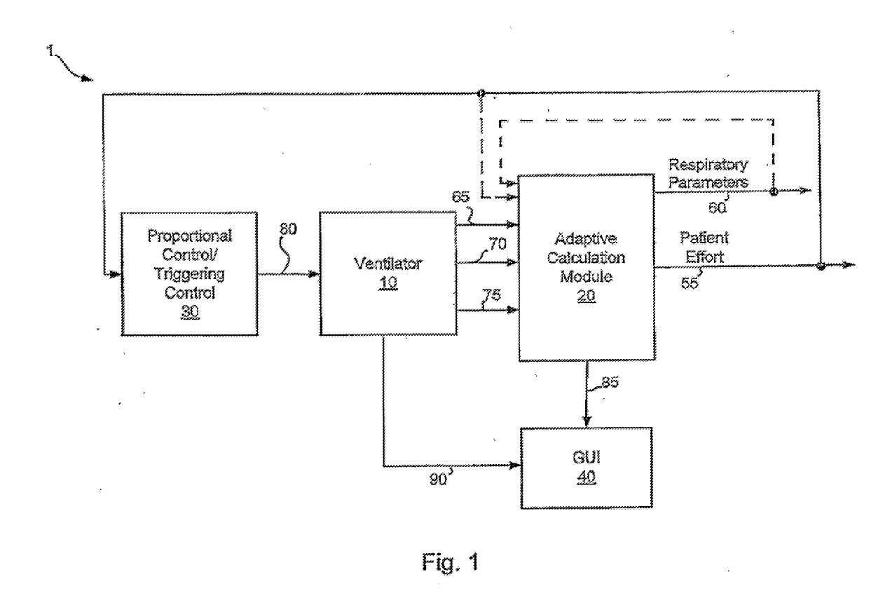

Turning to FIG. 1, a ventilation system 1 is shown in accordance with various embodiments of the present invention. Ventilation system 1 includes a ventilator 10, an adaptive calculation module 20, a graphical user interface 40, and a proportional and triggering control module 30. Ventilator 10 may be any ventilator known in the art that is capable of providing a measured pressure 65, a measured inlet flow 70 and a measured outlet flow 75. Adaptive calculation module 20 receives pressure 65, inlet flow 70 and outlet flow 75 and calculates an estimated patient effort 55 and estimated respiratory parameters 60. Patient effort 55 may be patient effort itself or some signal that is strongly correlated to patient effort. Signals correlated to patient effort are more fully discussed below. Respiratory parameters 60 may include a variety of parameters that are more fully described below. In an embodiment, the calculations performed by adaptive calculation module 20 may be adaptive in nature relying on previous interim values to generate updated respiratory parameters 60 and patient effort 55 estimates. In some embodiments, such interim values may include the patient effort 55 and/or the respiratory parameter estimates 60 as shown by dashed lines in FIG. 1. Alternatively (not shown), the previous interim values used by adaptive calculation module 20 may be composite parameters that do not directly correspond to any identifiable respiratory parameter (such as, for example, the covariance matrix and parameter vector discussed in greater detail below).

In the embodiment illustrated, patient effort 55 is provided to proportional and triggering control module 30. Based on patient effort 55, proportional and triggering control module 30 generates one or more control signals 80 that are provided to ventilator 10. In some embodiments, control signals 80 control the timing of gas delivery to a patient. In various embodiments, control signals 80 control the amount of gas to be delivered to a patient, where the amount of gas is in proportion to patient effort 55.

Ventilator 10 provides control signals 90 that drive graphical user interface 40. Graphical user interface 40 may be included as part of ventilator 10 to allow for interaction with a user including, but not limited to, receiving user commands and/or displaying data relevant to ventilator operation. In some embodiments, ventilator 10 may direct graphical user interface 40 to display information 85 provided by adaptive calculation module 20. Such information may include, but is not limited to, respiratory parameters 60 and/or patient effort 55 as is more fully discussed below.

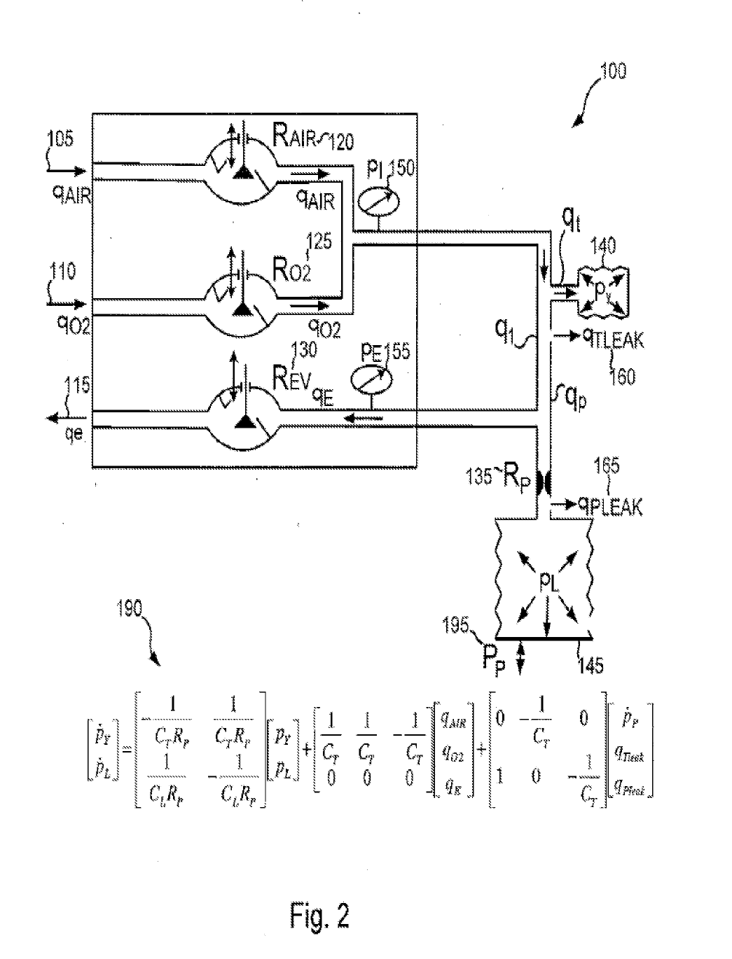

Various embodiments of the present invention utilize a parameterized dynamic model of a patient ventilator system to determine patient effort. A model of a ventilator system 100 is depicted in FIG. 2. Ventilator system 100 includes an inlet air flow 105 (q_{AIR}), an inlet Oxygen flow 110 (q_{O2}), and an outlet gas flow 115 (q_{R}). It should be noted that while ventilator system 100 shows two gas sources, Air and Oxygen, more or fewer inlet gas sources may be used in relation to different embodiments of the present invention. For example, it may be that only an Air source is used, or that in addition to the inlet Air source and the inlet Oxygen source, a Helium and/or Heliox source may be included. Based on the disclosure provided herein, one of ordinary skill in the art will recognize a variety of other gas sources that may be used in relation to different embodiments of the present invention.

Tubing, flow valves, and/or pressure monitors included in the system introduce some resistance to gas flow in ventilator system 100. In particular, an air resistance 120 (Rₐᵢᵣ), an Oxygen resistance 125 (R_{O2}), an exhalation resistance 130 (R_{EV}), and a patient resistance 135 (R_{P}) (i.e., some combination of trachea resistance and resistance in an endotracheal tube) are possible. A pressure sensor 150 measures the pressure (p₁) at the inlet at a location where the air flow and Oxygen flow is combined, and a pressure sensor 155 measures the pressure (p_{E}) in an exhalation output. It should be noted that pressure sensor 150 may be replaced by individual pressure sensors associated with respective inlet lines. The pressure (p_{Y}) at a location where inlet and outlet gases combine is represented as a baffles 140 (e.g., wye gas pressure), and the pressure (p_{L}) in the patient's lungs is represented by another baffles. In some embodiments of the present invention, p_{Y} is determined though use of a pressure measurement device mounted at or near the particular location corresponding to the pressure. In other embodiments of the present invention, p_{Y} is set equal to either p_{I} or p_{E} , while in other embodiments of the present invention, p_{Y} is set to the average of p_{I} and p_{E}. In any of the aforementioned three cases, p_{Y} is considered to be “directly measured” as it is either a measurement or is an average of other direct measurements. A gas flow associated with a leakage 160 (q_{Tleak}) in the tubing, and a gas flow associated with a leakage 165 (q_{Pleak}) in the patient are also identified. A patient effort value 195 (p_{P}) is shown as a force interacting with the force of moving gas in and out of a patient's lung.

Various equations may be used to describe the operation of ventilator system 100. For example, using the principle of conservation of mass, the various flow values (i.e., g_{AIR}, q_{O2}, q_{T} ³, q_{Tleak}, q_{Pleak}, q_{LUNG}, q₁) may be combined to yield the following three equations:

q_{LUNG}=q_{P}−q_{Pleak};

q₁−q_{P}−q_{E}=0; and

q_{AIR}+q_{O2}=q₁+q_{Tleak}+q_{T}.

Further, using the principle of equilibrium of forces, the pressures p_{Y}, p_{L}, and pₚ, and flows q_{T} and q_{L} can be combined in the following relationships:

$${p}_{Y} = \frac{1}{{C}_{T}} \int {q}_{T} d t , or {\dot{p}}_{Y} = \frac{1}{{C}_{T}} {q}_{T} ; and$$

$${p}_{p} - {p}_{L} = \frac{1}{{C}_{L}} \int {q}_{L} d t , or {\dot{p}}_{L} = {\dot{p}}_{p} - \frac{1}{{C}_{L}} {q}_{L} .$$

Finally, the relationship between pressure and flow can be used to derive the following equation based on ventilator system 100:

p_{Y}−p_{L}=R_{P}·q_{P},

By algebraically manipulating the aforementioned equations derived from ventilator system 100 and recasting the equations in a matrix form, the following parameterized model 190 is developed to characterize the operation of ventilator system 100 of FIG. 2:

$$[ \begin{matrix}{\dot{p}}_{Y} \\ {\dot{p}}_{L}\end{matrix} ] = [ \begin{matrix}- \frac{1}{{C}_{T} {R}_{P}} & \frac{1}{{C}_{T} {R}_{P}} \\ \frac{1}{{C}_{L} {R}_{P}} & - \frac{1}{{C}_{L} {R}_{P}}\end{matrix} ] [ \begin{matrix}{p}_{Y} \\ {p}_{L}\end{matrix} ] + [ \begin{matrix}\frac{1}{{C}_{T}} & \frac{1}{{C}_{T}} & - \frac{1}{{C}_{T}} \\ 0 & 0 & 0\end{matrix} ] [ \begin{matrix}{q}_{AIR} \\ {q}_{O 2} \\ {q}_{E}\end{matrix} ] + [ \begin{matrix}0 & - \frac{1}{C} & 0 \\ 1 & 0 & - \frac{1}{{C}_{T}}\end{matrix} ] [ \begin{matrix}{\dot{p}}_{P} \\ {q}_{Tleak} \\ {q}_{Pleak}\end{matrix} ] ,$$

where \dot{p}_{Y} is the first derivative of the pressure measured at the tubing branch, \dot{p}_{L} is the first derivative of the pressure in the patient's lung, \dot{p}_{P} is the first derivative of the patient effort, C_{T} represents tubing compliance, and C_{L} represents lung compliance. It should be noted that where more or fewer inlet gases are utilized, that parameterized model 190 may be modified to account for the different gases in accordance with other embodiments of the present invention.

Various embodiments of the present invention utilize parameterized model 190 to determine patient effort, p_{P}. In different embodiments of the present invention, assumptions may be made to simplify the calculation. In one particular embodiment of the present invention, leakage 160 may be assumed to exhibit the following linear relationship between the tubing leak flow and the pressure drop across an opening:

$${q}_{Tleak} = \frac{1}{{R}_{LEAK}} {p}_{y} = {λ}_{LEAK} {p}_{y} .$$

It should be noted that in other embodiments of the present invention, other assumptions about the relationship between the tubing leak flow and the pressure drop across an opening may be used. Relying on the aforementioned linear assumption for the tubing leak flow, parameterized model 190 may be reduced to the following model:

$$[ \begin{matrix}{\dot{p}}_{Y} \\ {\dot{p}}_{L}\end{matrix} ] = [ \begin{matrix}- \frac{1}{{C}_{T} {R}_{P}} - \frac{{λ}_{Tleak}}{{C}_{T}} & \frac{1}{{C}_{T} {R}_{P}} \\ \frac{1}{{C}_{L} {R}_{P}} & - \frac{1}{{C}_{L} {R}_{P}}\end{matrix} ] [ \begin{matrix}{p}_{Y} \\ {p}_{L}\end{matrix} ] + [ \begin{matrix}\frac{1}{{C}_{T}} & \frac{1}{{C}_{T}} & - \frac{1}{{C}_{T}} \\ 0 & 0 & 0\end{matrix} ] [ \begin{matrix}{q}_{AIR} \\ {q}_{O 2} \\ {q}_{E}\end{matrix} ] + [ \begin{matrix}0 & 0 \\ 1 & - \frac{1}{{C}_{L}}\end{matrix} ] [ \begin{matrix}{\dot{p}}_{p} \\ {q}_{Pleak}\end{matrix} ] ,$$

Based on the aforementioned parameterized model, the transfer function for p_{Y} is defined as follows:

$$\begin{matrix}{p}_{Y} ( s ) = \frac{{b}_{q} ( s )}{a ( s )} ( {q}_{AIR} ( s ) + {q}_{O 2} ( s ) - {q}_{E} ( s ) ) + \frac{{b}_{{p}_{p}} ( s )}{a ( s )} {\dot{p}}_{p} ( s ) + \frac{{b}_{Pleak} ( s )}{a ( s )} {q}_{Pleak} ( s ) \\ = \frac{{b}_{q} ( s )}{a ( s )} {q}_{N} ( s ) + \frac{{b}_{{p}_{p}} ( s )}{a ( s )} {\dot{p}}_{p} ( s ) + \frac{{b}_{Pleak} ( s )}{a ( s )} {q}_{Pleak} ( s ) ,\end{matrix}$$

where the instantaneous sum of each of the measured flows (e.g., q_{AIR}+q_{O2}−q_{R}) is denoted q_{N} for net flow.

$$\frac{{b}_{q} ( s )}{a ( s )} {q}_{N} ( s )$$

represents a transfer function from the net flow (q_{N}) to the output (p_{Y}),

$$\frac{{b}_{{p}_{p}} ( s )}{a ( s )} {\dot{p}}_{p} ( s )$$

represents a transfer function from the derivative of patient effort (\dot{p}_{P}) to the output (p_{Y}), and

$$\frac{{b}_{Pleak} ( s )}{a ( s )} {q}_{Pleak} ( s )$$

represents a transfer function from patient leakage (q_{Pleak}) to the output (p_{Y}). It should be noted that the first term in the preceding transfer function (i.e., the q_{N} term) is a transfer function related to a known, measured value, and the second term in the preceding transfer function (i.e., the p_{P} term) is a transfer function related to an unknown, adaptively estimated value. In some embodiments of the present invention, the third term (i.e., the q_{Pleak} term is assumed to be zero for the sake of simplification. Again, using the above mentioned parameterized model, the relationships between the transfer function coefficients and the system parameters are as follow:

$$\begin{matrix}a ( s ) = {s}^{2} + \frac{{C}_{L} + {C}_{T} + {C}_{L} {R}_{P} {λ}_{Tleak}}{{C}_{L} {C}_{T} {R}_{P}} s + \frac{{λ}_{Tleak}}{{C}_{L} {C}_{T} {R}_{P}} \\ = {s}^{2} + {a}_{1} s + {a}_{0} ,\end{matrix}$$

$$with$$

$${a}_{1} = \frac{{C}_{L} + {C}_{T} + {C}_{L} {R}_{P} {λ}_{Tleak}}{{C}_{L} {C}_{T} {R}_{P}} , \\ {a}_{0} = \frac{{λ}_{Tleak}}{{C}_{L} {C}_{T} {R}_{P}}$$

$${b}_{q} ( s ) = \frac{1}{{C}_{T}} s + \frac{1}{{C}_{L} {C}_{T} {R}_{P}} = {b}_{q 1} s + {b}_{q 0} , \\ with$$

$${b}_{q 1} = \frac{1}{{C}_{T}} , \\ {b}_{q 0} = \frac{1}{{C}_{L} {C}_{T} {R}_{P}}$$

$${b}_{{p}_{p}} ( s ) = \frac{1}{{C}_{T} {R}_{P}} = {b}_{{p}_{p 0}}$$

$${b}_{Pleak} ( s ) = - \frac{1}{{C}_{L} {C}_{T} {R}_{P}} = {b}_{Pleak 0}$$

From the forgoing, it is possible to derive a parameterized output model in a linear regression form. A first step in defining the parameterized linear regression output model includes defining an unknown parameter vector such as the following:

θ^{T}=[a₀ a₁ b_{q0} b_{q1}].

From the unknown parameter model, once estimated, all lumped parameters of ventilator system 100 (e.g., C_{T}, C_{L}, R_{P}, and λ_{LEAK}) may be recovered. Through algebraic manipulation of the transfer function for p_{Y} may be represented as:

$${p}_{Y} ( s ) \frac{{s}^{2}}{Λ ( s )} = - {p}_{Y} ( s ) \frac{( {a}_{1} s + {a}_{0} )}{Λ ( s )} + {b}_{q} ( s ) \frac{{q}_{N} ( s )}{Λ ( s )} + {b}_{{p}_{p}} ( s ) \frac{{\dot{p}}_{p} ( s )}{Λ ( s )} + {b}_{Pleak} ( s ) \frac{{q}_{Pleak} ( s )}{Λ ( s )} .$$

In this case, the pressure

$${p}_{Y} ( s ) \frac{{s}^{2}}{Λ ( s )}$$

represents pressure p_{Y} (s) after filtering through a proper filter,

$$\frac{{s}^{2}}{Λ ( s )} .$$

Such a proper filter relies on a polynomial Λ(s) that is the same or of higher order than s² (e.g., s², s³ , s⁴ . . . ). By assuming that patient leakage (q_{Pleak}) is zero, a compact linear regression form of the input to output relationship corresponding to parameterized model 190 of ventilation system 100 is represented as:

$$z = {Θ}^{T} ϕ + {ϕ}_{d}$$

$$z = {p}_{Y} ( s ) \frac{{s}^{2}}{Λ ( s )}$$

$${Θ}^{T} = [ \begin{matrix}{a}_{0} & {a}_{1} & {b}_{q 0} & {b}_{q 1}\end{matrix} ]$$

$${ϕ}^{T} = [ \begin{matrix}- \frac{{p}_{Y} ( s )}{Λ ( s )} & - \frac{{p}_{Y} ( s ) s}{Λ ( s )} & \frac{{q}_{N} ( s )}{Λ ( s )} & \frac{{q}_{N} ( s ) s}{Λ ( s )}\end{matrix} ]$$

$${ϕ}_{d} = {b}_{{p}_{p}} ( s ) \frac{{\dot{p}}_{p} ( s )}{Λ ( s )}$$

where z is the output pressure value, φ^{T} is the regression vector representing a collection of known signals, and φ_{d} is filtered patient effort.

In this case, use of standard linear regression to estimate the system parameters θ^{T}=[a₀ a₁ b_{q0} b_{q1}] is not possible as φ_{d} is unknown. By inspecting the unknown term

$${ϕ}_{d} = {b}_{{p}_{p}} ( s ) \frac{{\dot{p}}_{p} ( s )}{Λ ( s )} ,$$

and understanding that the derivative of patient effort (\dot{p}_{P}) is a bounded signal, that the filter (Λ(s)) is a stable polynomial, and

$$\frac{{b}_{{p}_{p}} ( s )}{Λ ( s )}$$

is a proper linear filter, it is apparent that the unknown filtered patient effort (i.e., φ_{d}) is a smooth signal. Based on this understanding, the value of the unknown filtered patient effort at any time t can be approximated by its value at the time t-dt, where dt represents an infinitesimal or finite, but small amount of time:

φ_{d}≈φ_{d}e^{-s·dt}=e^{-s·dt}(z−θ^{T}φ).

In some embodiments of the present invention, dt is five milliseconds or less. The aforementioned approximation represents a reasonable guess, or prediction, of the unknown filtered patient effort signal at time t that may be used in calculating respiratory parameters, and thereafter in calculating patient effort. This reasonable guess can be used to determine the predicted value (\hat{z}) of the system output (z) can be defined in accordance with the following equation:

\hat{z}=θ^{T}φ+e^{-s·dt}(z−θ^{T}φ)=θ^{T}(φ−e^{-s·dt}φ)+e^{-s·dt}z.

From this definition, the parametric identification problem can be solved through formulation of the following problem: Given φ(t), z(t), find

$$Θ = arg ⌊ {min}_{Θ} J ( z - \hat{z} ) ⌋ ,$$

where J( ) is a convex (e.g., ( )²) function of θ. From this point, one of a number of mathematical solutions may be applied to resolve the problem. As one example, a modified recursive least squares method may be used. More detail related to a non-modified mathematical implementation of such an approach is more fully described in one or both of (1) Lennart Ljung, “System Identification, Theory for the User”, Second Edition, Prentice Hall, 1999 (ISBN 0-13-656695-2) and (2) Petros Ioannou and Jing Sun, Robust Adaptive Control, Prentice Hall, 1995 (ISBN 9780134391007). Both of the aforementioned references are incorporated herein by reference for all purposes.

In implementing a modified recursive least squares method, a prediction error (ε) is first normalized and signals are adopted for the normalized signals as set forth in the following equation:

$$\begin{matrix}ɛ = \frac{z - \hat{z} ( t )}{{m}^{2}} \\ = \frac{z - {Θ}^{T} ( ϕ - {e}^{- s \cdot dt} ϕ ) - {e}^{- s \cdot dt} z}{{m}^{2}} \\ = \frac{z ( 1 - {e}^{- s \cdot dt} ) - {Θ}^{T} ϕ ( 1 - {e}^{- s \cdot dt} )}{{m}^{2}} \\ = \frac{\tilde{z} - {Θ}^{T} \tilde{ϕ}}{{m}^{2}} \\ \tilde{z} = z ( 1 - {e}^{- s \cdot dt} ) \\ \tilde{ϕ} = ϕ ( 1 - {e}^{- s \cdot dt} ) \\ {m}^{2} = 1 + {\tilde{ϕ}}^{T} \tilde{ϕ}\end{matrix}$$

where ε is the normalized prediction error, \tilde{z} and \tilde{φ} are the differences of the output and regressor respectively corresponding to the time interval dt, and in is the normalization signal. In addition, a modified function J( ) (referred to as a cost function) is adopted in accordance with the following equation:

$$J ( Θ ( t ) ) = \frac{1}{2} {\int }_{0}^{t} {e}^{- β ( t - τ )} \frac{{( \tilde{z} - {Θ}^{T} \tilde{ϕ} )}^{2}}{{m}^{2}} d τ + \frac{1}{2} {{e}^{- β l} ( Θ - {Θ}_{0} )}^{T} {Q}_{0} ( Θ - {Θ}_{0} ) ,$$

where β>0 and Q₀≧0 are referred to as a forgetting factor and a penalty matrix. Based on this, the following stationary conditions must be met at the solution θ:

$$\begin{matrix}\frac{\partial }{\partial Θ} = J ( Θ ( t ) ) \\ = {e}^{- β l} {Q}_{0} ( Θ - {Θ}_{0} ) - {\int }_{0}^{t} {e}^{- β ( t - τ )} \frac{\tilde{z} \tilde{ϕ}}{{m}^{2}} d τ + {\int }_{0}^{t} {e}^{- β ( t - τ )} \frac{\tilde{ϕ} {\tilde{ϕ}}^{T}}{{m}^{2}} d τΘ = \\ = [ {e}^{- β l} {Q}_{0} + {\int }_{0}^{t} {e}^{- β ( t - τ )} \frac{\tilde{ϕ} {\tilde{ϕ}}^{T}}{{m}^{2}} d τ ] Θ - \\ [ {e}^{- β l} {Q}_{0} {Θ}_{0} + {\int }_{0}^{t} {e}^{- β ( t - τ )} \frac{\tilde{z} \tilde{ϕ}}{{m}^{2}} d τ ] = \\ = {P}^{- 1} Θ - [ {e}^{- β l} {Q}_{0} {Θ}_{0} + {\int }_{0}^{t} {e}^{- β ( t - τ )} \frac{\tilde{z} \tilde{ϕ}}{{m}^{2}} d τ ] = 0\end{matrix}$$

Thus, θ can be found non-recursively as:

$$Θ = P [ {e}^{- β l} {Q}_{0} ( Θ - {Θ}_{0} ) + {\int }_{0}^{t} {e}^{- β ( t - τ )} \frac{\tilde{z} \tilde{ϕ}}{{m}^{2}} d τ ] ,$$

where:

$$P = {[ {e}^{- β l} {Q}_{0} + {\int }_{0}^{t} {e}^{- β ( t - τ )} \frac{\tilde{ϕ} {\tilde{ϕ}}^{T}}{{m}^{2}} d τ ]}^{- 1} .$$

Matrix P and vector θ satisfy the following two differential equations which complete the definition of the recursive algorithm that can be used to solve the parameter identification problem:

$$\dot{P} = β P - P \frac{\tilde{ϕ} {\tilde{ϕ}}^{T}}{{m}^{2}} P , \\ P ( 0 ) = {P}_{0} = {Q}_{0}^{- 1} ,$$

\dot{θ}=Pε\hat{φ}

where ε is the normalized error or difference between the last measured values and current measured values,

In the following discussion, methods are described that can be used to indirectly estimate a current value of patient effort in real time. In addition, it is demonstrated how various combinations of the above mentioned interim values (e.g., signals internal the transfer function) explained above possess a significant level of correlation with the unmeasured patient effort. Because of the correlation, the interim values may be used to characterize patient effort with a reasonable degree of accuracy.

From the relationships established above, it is clear that:

$${ϕ}_{d} = ( z - {Θ}^{T} ϕ ) = {b}_{{p}_{p}} ( s ) \frac{{\dot{p}}_{p} ( s )}{Λ ( s )} .$$

By choosing an appropriate filter,

$$\frac{1}{Π ( s )} ,$$

that yields

$$\frac{Λ ( s )}{{b}_{{p}_{p}} ( s )} \frac{1}{Π ( s )} ,$$

an estimate of the derivative of patient effort ({circumflex over (\dot{p}_{P}) of the real derivative of patient effort (\dot{p}_{P}) can be computed as follows:

$${{p}^{\hat{.}}}_{p} ( s ) = ( z - {Θ}^{T} ϕ ) \frac{Λ ( s )}{{b}_{{p}_{p}} ( s )} \frac{1}{Π ( s )} .$$

Based on the following equation, it is apparent that a prediction error signal, z−\hat{z}, is correlated with the patient effort signal, \dot{p}_{P}, and the filtered version thereof, φ_{d}:

$$\begin{matrix}z - \hat{z} = {Θ}^{T} ϕ + {ϕ}_{d} - ( {Θ}^{T} ϕ + {e}^{- s \cdot dt} ( z - {Θ}^{T} ϕ ) ) \\ = {ϕ}_{d} - {e}^{- s \cdot dt} {ϕ}_{d} \\ = d \frac{{ϕ}_{d} ( t - d t )}{d t} d t .\end{matrix}$$

Using the transfer function defined above and the current estimate of the parameter vector θ, a prediction (\hat{p}_{y}) of the current pressure in the tubing (p_{y}) is represented by the following equation:

$${\hat{p}}_{Y} ( s , Θ ) = \frac{{b}_{q} ( s , Θ )}{a ( s , Θ )} {q}_{N} ( s ) .$$

From this, the prediction error may be described by the following equation:

$${p}_{y} - {\hat{p}}_{y} = \frac{{b}_{{p}_{p}} ( s )}{a ( s )} {\dot{p}}_{p} ( s ) ,$$

Which is a filtered version of the derivative of patient effort (\dot{p}_{P}). Moreover, if the ventilation system is characterized by the absence of tubing leaks (i.e., assume λ_{LEAK}=0), then the prediction error, p_{y}−\hat{p}_{y}, resembles the patient effort signal (p_{P}) as the transfer function

$$\frac{{b}_{{p}_{p}} ( s )}{a ( s )}$$

is an integration function.

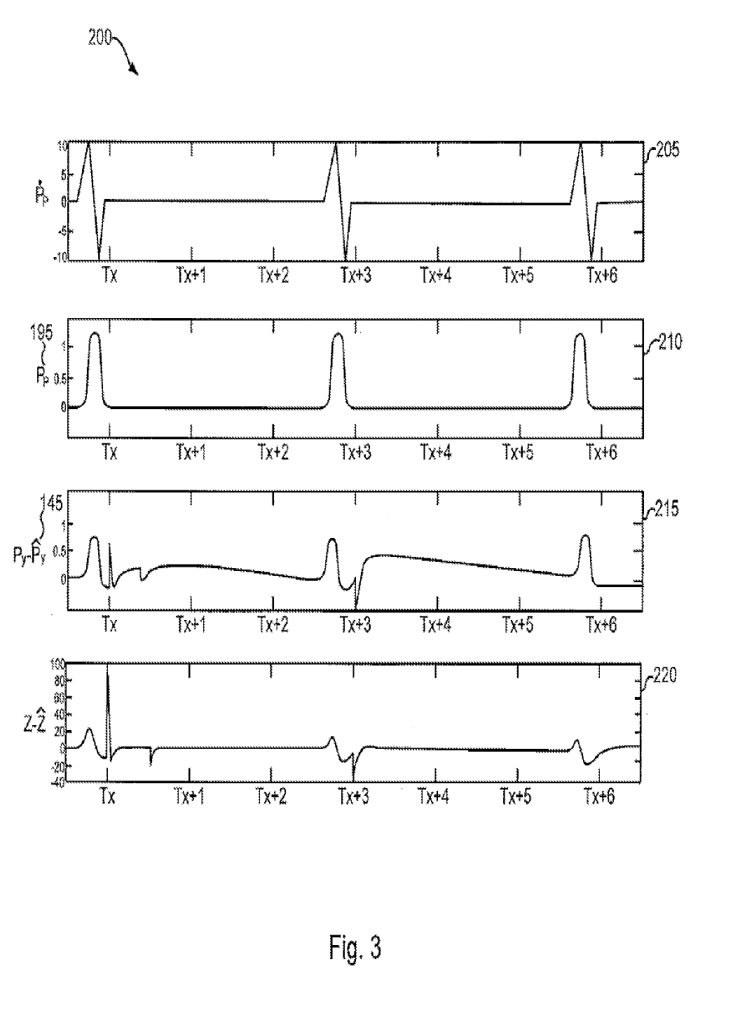

The aforementioned equations describe relationships between patient effort (i.e., p_{P} and/or \dot{p}ₚ), and accurately obtainable flow and pressure measurements. FIG. 3 graphically depicts the exemplary correlation between patient effort (i.e., p_{P} and/or \dot{p}_{P}) and exemplary signals internal to the previously described algorithm. As shown, a timing diagram 210 depicts patient effort (p_{P}) as a function of time. A timing diagram 205 depicts the first derivative of patient effort (\dot{p}_{P}) as a function of time. A timing diagram 215 depicts p_{y}−\hat{p}_{y}, and a timing diagram 220 depicts z−\hat{z}. The magnitude of each of p_{P}, \dot{p}_{P}, p_{Y}−\hat{p}^{y} and z−\hat{z} is represented in centimeters of H₂O. As would be expected based on the analysis provided above, there is a strong correlation between patient effort (p_{P}) depicted in diagram 210 and the signal p_{y}−\hat{p}_{y} depicted in diagram 215. Similarly, diagrams 205 and 220 demonstrate a strong correlation between the first derivative of the patient effort, \dot{p}ₚ, and the signal z−\hat{z}. Thus, the reconstructed signals can be used to predict the otherwise unknown signals \dot{p}ₚ and pₚ. It should be noted that the results are merely exemplary, and that based on the disclosure provided herein, one of ordinary skill in the art will recognize a variety of different signals and their delayed versions that may be achieved through use of different embodiments of the present invention to characterize the unknown patient effort signals and the derivatives thereof.

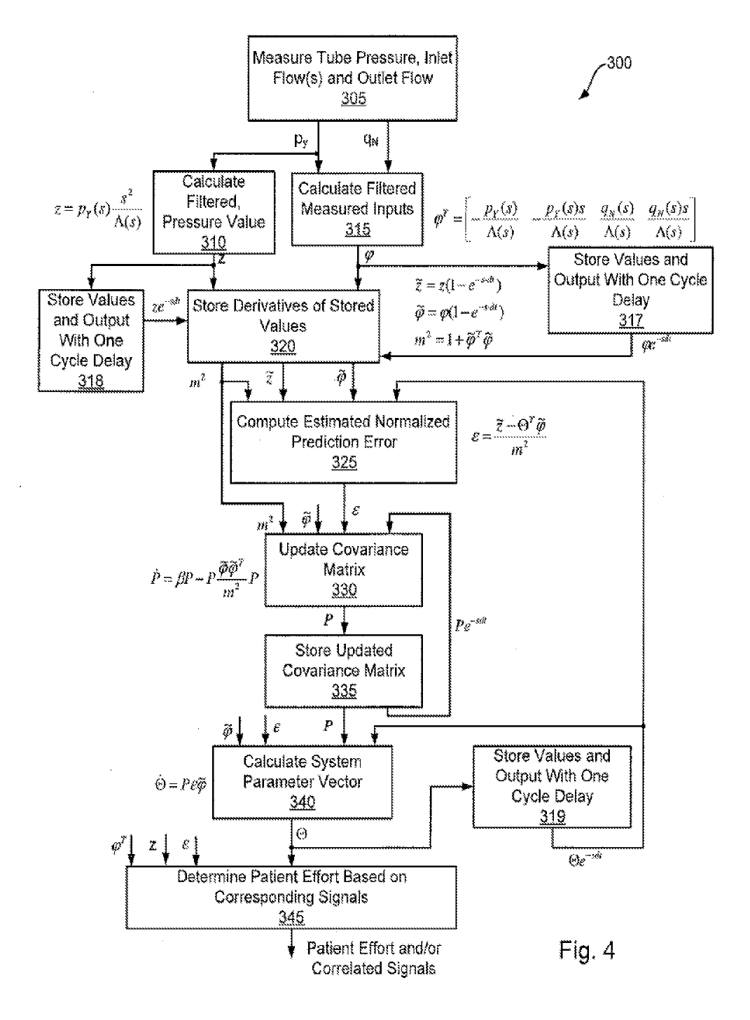

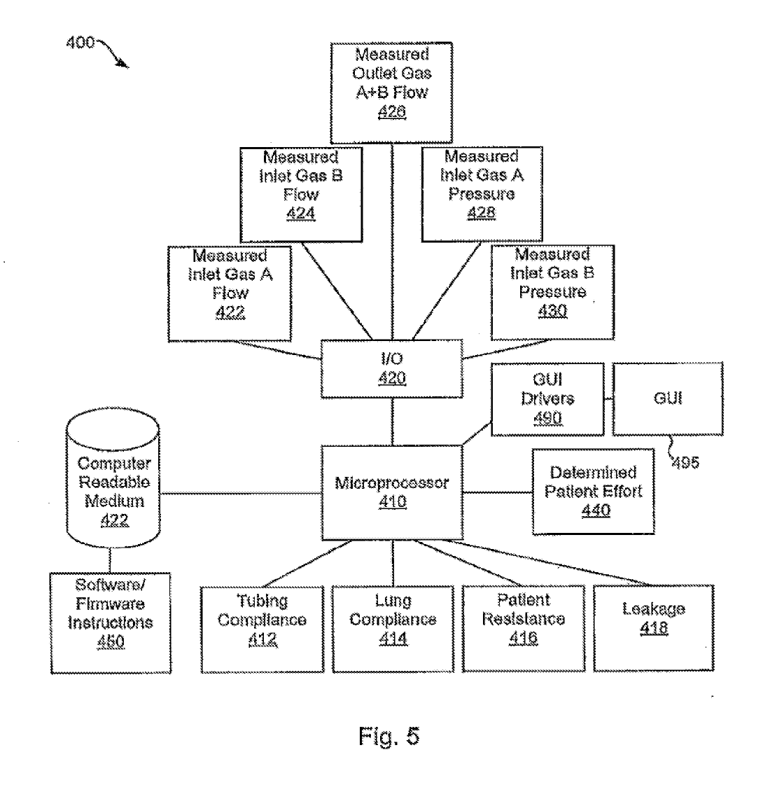

Turning to FIG. 4, a flow diagram 300 depicts a method in accordance with some embodiments of the present invention for determining patient effort. A ventilator system is provided that includes a ventilator that is coupled to a subject using various tubing. The ventilator receives one or more inlet gas flows and includes an outlet gas flow in addition to an inlet/outlet to the subject. Following flow diagram 300, pressure in the tubing (p_{y}) is measured along with the inlet flow(s) and the outlet flow to generate a net flow (qₙ) (block 305). The pressure value (p_{y}) is filtered and provided as an output (z) (block 310), and the pressure (p_{y}) and net flow value (qₙ) are filtered and combined in a regression vector (φ^{T}) (block 315). Differences and/or derivatives of the aforementioned values (i.e., z and φ^{T}) are calculated to generate outputs m² , \tilde{z} and \tilde{φ} (block 320). In addition, time delayed versions of z (i.e., ze^{-sdt}) and \tilde{φ} (i.e., \tilde{φ}e^{-sdt}) are created (blocks 317, 318). m², \tilde{z}, \tilde{φ} and θ^{T} are combined to generate an estimated normalized prediction error (ε) (block 325); and m², \tilde{φ} and ε are used along with a previously computed covariance matrix (P₀) to calculate an updated covariance matrix (P) (block 330). The newly calculated covariance matrix (P) is stored and maintained as the previously computed covariance (P₀) for use in later updating of the covariance matrix (block 335). The updated covariance matrix (P) is used along with the previously computed ε and \tilde{φ} values to calculate an updated system parameter vector (θ) (block 340). In addition, a time delayed version of θ (i.e., θe^{-sdt}) generated (block 319). As discussed above, the system parameter vector (θ) incorporates various system parameters including, for example, tubing compliance (C_{T}), lung compliance (C_{L}), lumped resistance (R_{P}), and leakage (λ_{LEAK}).

During the above mentioned processing (blocks 305-340), various of the interim values may be used either separately or in combination to estimate patient effort (block 345). For example, as depicted in FIG. 3 above, z correlates to patient effort. Further, as z may be calculated using other constituent elements, the constituent elements may also be used to estimate patient effort. Based on the disclosure provided herein, one of ordinary skill in the art will recognize other uses of the constituent elements to predict patient effort.

Turning to FIG. 5, a microprocessor based system 400 for determining patient effort is depicted in accordance with various embodiments of the present invention. System 400 includes a microprocessor 410 communicably coupled to a computer readable medium 460. Microprocessor 410 may be any processor known in the art that is capable of receiving various input values, and executing software or firmware instructions to provide an output based on the input values. Computer readable medium 460 may be any media capable of storing instructions that are executable by microprocessor 410. Based on the disclosure provided herein, one of ordinary skill in the art will recognize a variety of processors that may be used in relation to different embodiments of the present invention. As just some examples, computer readable medium 460 may be a hard disk drive, a tape drive, a portable solid state memory, a CD ROM, a RAM, combinations of the aforementioned, or the like. Based on the disclosure provided herein, one of ordinary skill in the art will recognize a variety of media and combinations of the media that may be used in relation to different embodiments of the present invention.

Instructions 450 when executed cause microprocessor 410 to receive various I/O via an I/O interface 420. The received I/O include measured inlet gas flows 422, 424, and a measured outlet gas flow 426. In some cases, the measured inlet gas flows measure the flow of Air and Oxygen, respectively. It should be noted that more or fewer than two inlet gas flows may be measured depending upon the particular embodiment of the present invention. Outlet gas flow 426 measures the gas flow being exhaled from system 400. Further, the received I/O include measured inlet gas pressures 428, 430 associated with the respective inlet gas flows 422, 424. It should be noted that where there are more or fewer inlet gas flows that the I/O may include more or fewer measured gas pressure inputs. Further, in some embodiments of the present invention, a single gas pressure input may be provided in place of inlet gas pressures 428, 430 where a single gas pressure sensor is placed in system 400 at a location that allows it to provide a pressure value that effectively combines inlet gas pressures 428, 430. Further, instructions 450 when executed cause microprocessor 410 to implement a patient effort algorithm using the I/O received via I/O interface 420, and providing a patient effort output 440. Such a patient effort algorithm may be, but is not limited to, the patient effort algorithms discussed above in relation to FIG. 2 and FIG. 4. As part of implementing the patient effort algorithm, instructions 450 cause microprocessor 410 to calculate a variety of otherwise unknown system parameters including, but not limited to, tubing compliance 412 (C_{T}), lung compliance 414 (C_{L}), lumped resistance 416 (R_{P}), and leakage 418 ( λ_{LEAK}). The aforementioned system parameters may be used in a variety of interim calculations with the results of one or more of the interim calculations providing results that are predictive of patient effort output 440.

In addition, microprocessor based system 400 may include a graphical user interface driver 490 and a graphical user interface 495. Graphical user interface 495 may be any interface that provides for graphically portraying information from microprocessor based system 400 to a user. Thus, graphical user interface 495 may be any display known in the art. In some cases, graphical user interface 495 may further include an ability to receive input from a user. The ability to receive input may be provided by, for example, a touch screen capability, a keyboard, a mouse, and/or the like deployed in association with graphical user interface 495. Graphical user interface driver 490 may be any circuit, system or device known in the art that is capable of converting information from microprocessor based system 400 into graphical information displayable via graphical user interface 495.

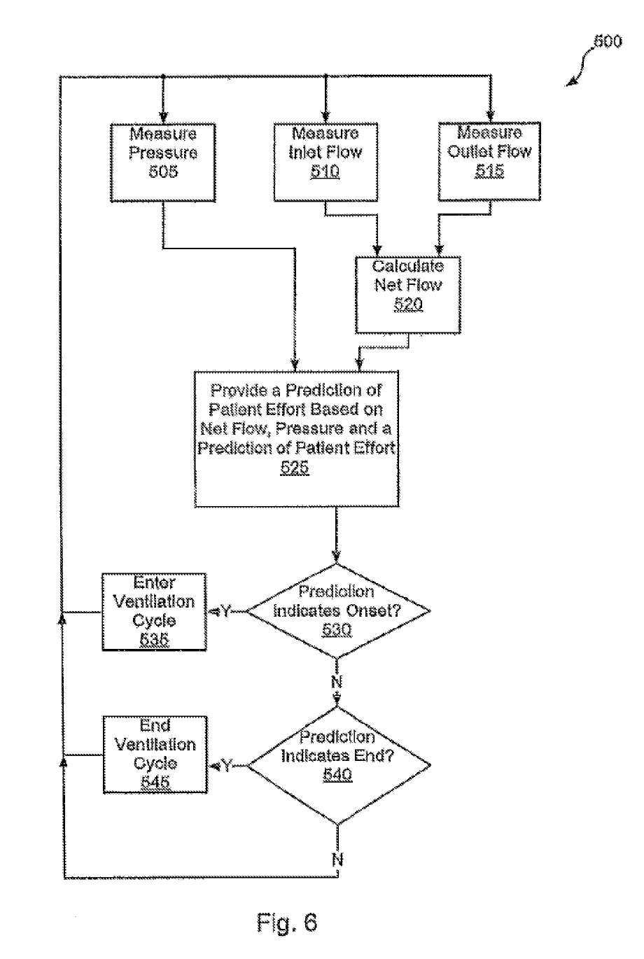

FIG. 6 is a flow diagram 500 depicting a method in accordance with some embodiments of the present invention for triggering a ventilation cycle. Following flow diagram 500, a pressure is measured (block 505), an inlet flow is measured (block 510), and an outlet flow is measured (block 515). In some cases, the pressure is measured in a tube connecting a ventilator to a person being ventilated. In some cases, the pressure is measured near a gas inlet and/or near a gas outlet. In other cases, the pressure is measured near a junction of the gas inlet with the gas outlet. In various cases, the pressure measurement is a single point pressure measurement, while in other cases the pressure measurement is a multiple point pressure measurement and the measured pressure is a mathematical combination of two or more pressure measurements. Measuring the inlet flow may include measuring the flow of a single gas, or measuring the flows of two or more gases and aggregating the multiple flow values. Measuring the outlet flow may include, but is not limited to, measuring the flow of gas at the outlet of the ventilation system. The outlet flow is subtracted from the inlet flow at a particular instance to generate an instantaneous net flow (block 520).

The net flow and measured pressure for a given instant are used to calculate an updated prediction of patient effort (block 525). This process may be done using the approach discussed above in relation to FIG. 4. It is then determined whether the updated prediction of patient effort indicates an onset condition (block 530). Where an onset condition is indicated (block 530), a ventilation cycle is triggered to begin (block 535). As an example, the updated prediction of patient effort may be the filtered patient effort signal (φ_{d}) that was discussed above. The filtered patient effort signal is a function of the derivative of patient effort (\dot{p}ₚ) as set forth in the following equation:

$${ϕ}_{d} = {b}_{{p}_{p}} ( s ) \frac{{\dot{p}}_{p} ( s )}{Λ ( s )} .$$

Thus, the filtered patient effort signal is expected to be negative when the actual patient effort (pₚ) is decreasing. Therefore, the onset of inspiration is indicated when the filtered patient effort signal becomes less than zero (e.g., exhibits a negative zero crossing where the signal transitions from a positive value to a negative value). This indicator may be used to synchronize the onset of a ventilation cycle with patient effort. Such synchrony results in improved patient ventilation. In some cases, a ventilation cycle is triggered to begin once the filtered patient effort signal is less than zero. In other cases, a ventilation cycle is triggered to begin once the filtered patient effort signal reaches a predefined negative threshold value or positive threshold value. It should be noted that while the filtered patient effort signal is used in the preceding example, that one or more other signals may be similarly used. For example, prediction error signal, z−\hat{z} may also be used as it is similarly correlated with actual patient effort. Based on the disclosure provided herein, one of ordinary skill in the art will recognize a variety of other signals that may be used to initiate a ventilation cycle.

Alternatively, it is determined whether the updated prediction of patient effort indicates an end condition (block 540). Where an end condition is indicated (block 540), a previously started ventilation cycle is triggered to terminate (block 545). As an example, the updated prediction of patient effort may be the same filtered patient effort signal used to trigger the onset of inspiration. As the filtered patient effort signal is a function of the derivative of patient effort, the end of inspiration is indicated when the filtered patient effort signal becomes greater than zero (e.g., exhibits a positive zero crossing where the signal transitions from a negative value to a positive value). Such an indicator may be used to synchronize the termination of a ventilation cycle with patient effort, and thereby provide improved patient ventilation. In some cases, a ventilation cycle is triggered to end once the filtered patient effort signal is greater than zero. In other cases, a ventilation cycle is triggered to end once the filtered patient effort signal reaches a predefined negative threshold value or positive threshold value. Again, it should be noted that while the filtered patient effort signal is used in the preceding example, that one or more other signals may be similarly used. For example, prediction error signal, z−\hat{z} may also be used as it is similarly correlated with actual patient effort. Based on the disclosure provided herein, one of ordinary skill in the art will recognize a variety of other signals that may be used to terminate a ventilation cycle.

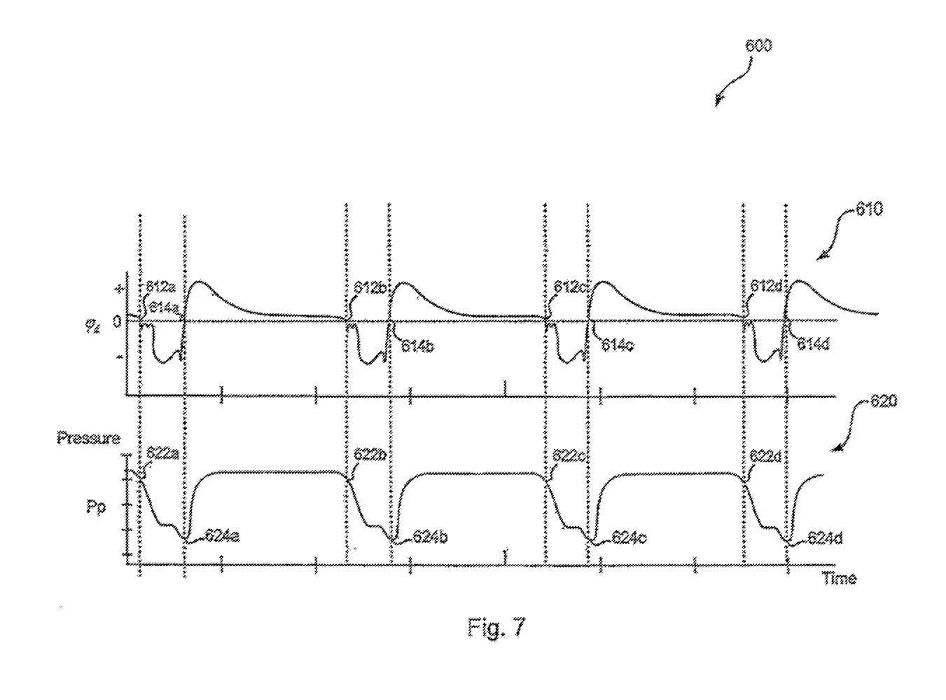

Turning to FIG. 7, a timing diagram 600 shows the process of triggering multiple ventilation cycles based on a proxy of patient effort. In this case, the proxy of patient effort is the filtered patient effort signal (φ_{d}) 610. An actual patient effort signal (Pₚ) 620 is shown to demonstrate the synchrony achievable using different embodiments of the present invention. It should be noted that while filtered patient effort signal 610 is shown as the ventilation trigger, that one or more other signals may be similarly used. For example, prediction error signal, z−\hat{z}, may also be used as it is similarly correlated with actual patient effort. Based on the disclosure provided herein, one of ordinary skill in the art will recognize a variety of other signals that may be used to effectuate triggering.

As shown, the transition of filtered patient effort signal 610 through a negative zero crossing point 612a corresponds to the beginning of an actual patient inspiration effort 622a. A subsequent positive zero crossing point 614a corresponds to the onset of exhalation 624a. This process is depicting for a number of ventilation cycles. Consistent with timing diagram 600, a positive zero crossing of filtered patient effort signal 610 may be used to trigger the beginning of a ventilation cycle, and a negative zero crossing of filtered patient effort signal 610 may be used to trigger the end of a ventilation cycle.

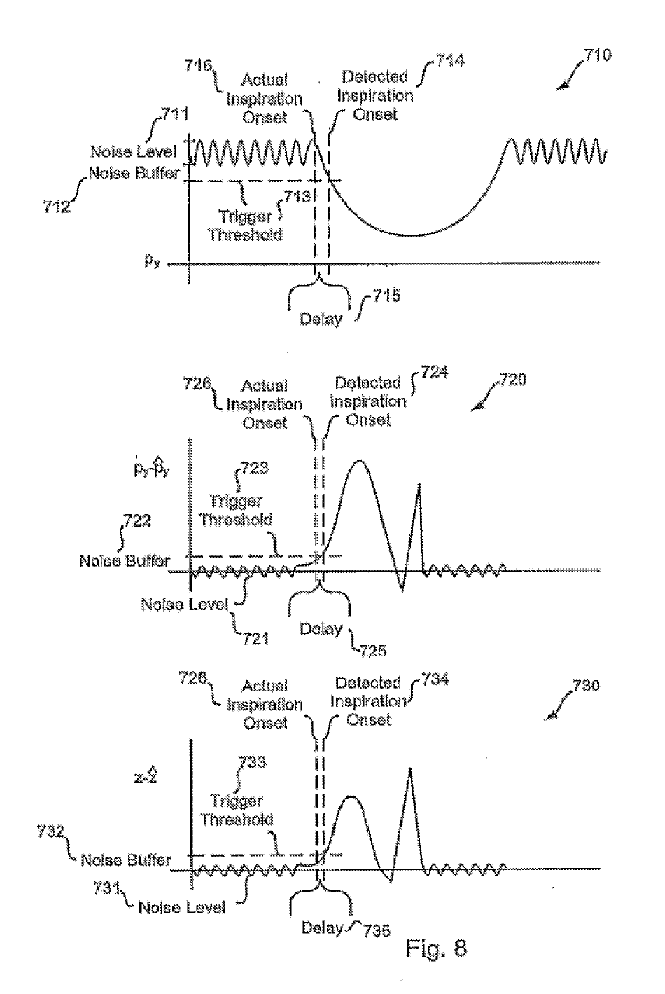

FIG. 8 includes a timing diagram 710 showing a process of triggering off of a pressure sensor corresponding to p_{y}, a timing diagram 720 showing a process of triggering off of an estimated patient effort signal, p_{y}−\hat{p}_{y}, and a timing diagram 730 showing a process of triggering off of another signal correlated to patient effort, z−\hat{z}. As shown by timing diagram 710, the pressure sensor exhibits a noise level 711 with a trigger threshold 713 set a noise buffer amount 712 below the expected noise level 711 to avoid false triggering. As shown, the pressure corresponding to p_{y} eventually drops below trigger threshold 713 resulting in a detected inspiration onset 714 (represented a vertical dashed line). Detected inspiration threshold 714 occurs a delay period 715 after an actual inspiration onset 716 (represented by a vertical dashed line). As can be seen from timing diagram 710, the magnitude of delay period 715 is a function of noise level 711 and noise buffer amount 712.

Noise associated with a pressure measurement is not necessarily correlated with that associated with flow measurements. By combining information derived from both pressure and flow measurements in the development of an estimated patient effort signal, the amount of noise expected is typically reduced when compared with the noise expected when only a single measurement is used. A noise buffer amount is often chosen based on the magnitude of expected noise. Thus, in some embodiments of the present invention, both the expected noise level and noise buffer amount are less than that exhibited in single measurement systems. The reduction of these variables allows for a detected inspiration that is correlated more closely in time with an actual inspiration onset. Timing diagrams 720, 730 graphically depict such a reduced trigger delay.

Following timing diagram 720, the estimated patient effort signal, p_{y}−\hat{p}_{y}, exhibits a relatively small noise level 721 with a trigger threshold 723 set a noise buffer amount 722 above the expected noise level 721 to avoid false triggering. As shown, the estimated patient effort signal eventually exceeds trigger threshold 723 resulting in a detected inspiration onset 724 (represented a vertical dashed line). Detected inspiration onset 724 occurs a delay period 725 after an actual inspiration onset 726 (represented by a vertical dashed line). Delay period 725 is less than that which results when only a single point of measurement is used. Similarly, following timing diagram 730, the estimated patient effort signal, z−\hat{z}, exhibits a relatively small noise level 731 with a trigger threshold 733 set a noise buffer amount 732 above the expected noise level 731 to avoid false triggering. As shown, the estimated patient effort signal eventually exceeds trigger threshold 733 resulting in a detected inspiration onset 734 (represented a vertical dashed line). Detected inspiration onset 734 occurs a delay period 735 after an actual inspiration onset 736 (represented by a vertical dashed line). Delay period 735 is less than that which results when only a single point of measurement is used.

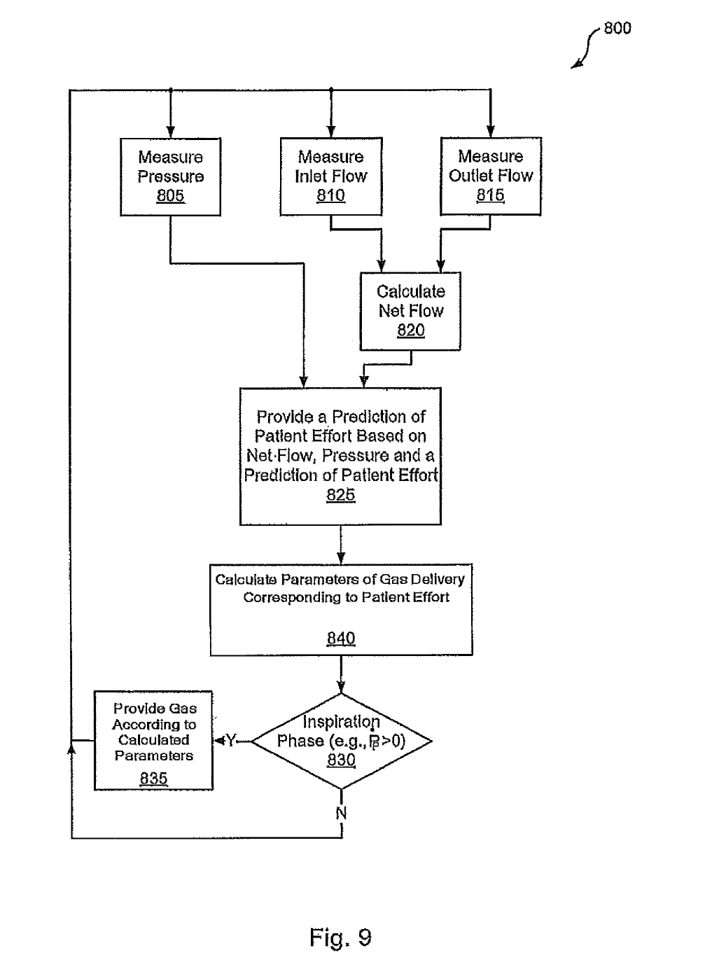

Turning to FIG. 9, a flow diagram 800 shows a method for providing ventilation in proportion to patient effort in accordance with various embodiments of the present invention. Following flow diagram 800, a pressure is measured (block 805), an inlet flow is measured (block 810), and an outlet flow is measured (block 815). In some cases, the pressure is measured in a tube connecting a ventilator to a person being ventilated. In some cases, the pressure is measured near a gas inlet and/or near a gas outlet. In other cases, the pressure is measured near a junction of the gas inlet with the gas outlet. In various cases, the pressure measurement is a single point pressure measurement, while in other cases the pressure measurement is a multiple point pressure measurement and the measured pressure is a mathematical combination of two or more pressure measurements. Measuring the inlet flow may include measuring the flow of a single gas, or measuring the flows of two or more gases and aggregating the multiple flow values. Measuring the outlet flow may include, but is not limited to, measuring the flow of gas at the outlet of the ventilation system. The outlet flow is subtracted from the inlet flow at a particular instance to generate an instantaneous net flow (block 820).

The net flow and measured pressure for a given instant are used to calculate an updated prediction of patient effort (block 825). This process may be done using the approach discussed above in relation to FIG. 4. Desired gas delivery parameter(s) of gas to be delivered by the ventilator an instant corresponding to the calculated patient effort is/are then calculated (block 840). In some embodiments of the present invention, the gas delivery parameters are flow and/or pressure. In this case, a desired pressure and flow of gas delivery are each a function of patient effort. For example, where patient effort is determined to be a value at an instant x described by a function f(x), then the calculated pressure may be described at an instant using the function g(f(x)) and the calculated flow at an instant may be described by the function h(f(x)). In one particular embodiment of the present invention, the function g and the function h are each constant multipliers. In such a case, the calculated pressure at an instant x is k₁f (x) and the calculated flow at the instant x is k₂f (x), where k₁ is the constant corresponding to pressure and k₂ is the constant corresponding to flow. Based on the disclosure provided herein, one of ordinary skill in the art will recognize other functions g functions h that may be used in relation to different embodiments of the present invention. The pressure used as a metric for delivering gas may be, but is not limited to, wye pressure or patient lung pressure. The flow used as a metric for delivering gas may be, but is not limited to, patient lung flow or inlet gas flow.

It is then determined whether the updated prediction of patient effort indicates an inspiration phase (block 830). In some embodiments of the present invention, an inspiration phase is indicated where the derivative of patient effort \dot{p}ₚ is greater than zero. Where an inspiration phase is indicated (block 830), gas is delivered to a recipient in accordance with the gas delivery parameters previously calculated (block 835). Again, gas delivery parameters may include, but are not limited to, pressures and flows of gas or gas components (e.g., oxygen, air, nitrogen, helium, etc.) to be delivered to a patient. Otherwise, where an inspiration phase is not indicated (block 830), gas delivery is not provided. Such an approach provides for gas delivery at a rate and/or pressure as a function of the patient's effort. Such an approach provides for increased patient comfort as well as less interference with a patient's own attempts at breathing.

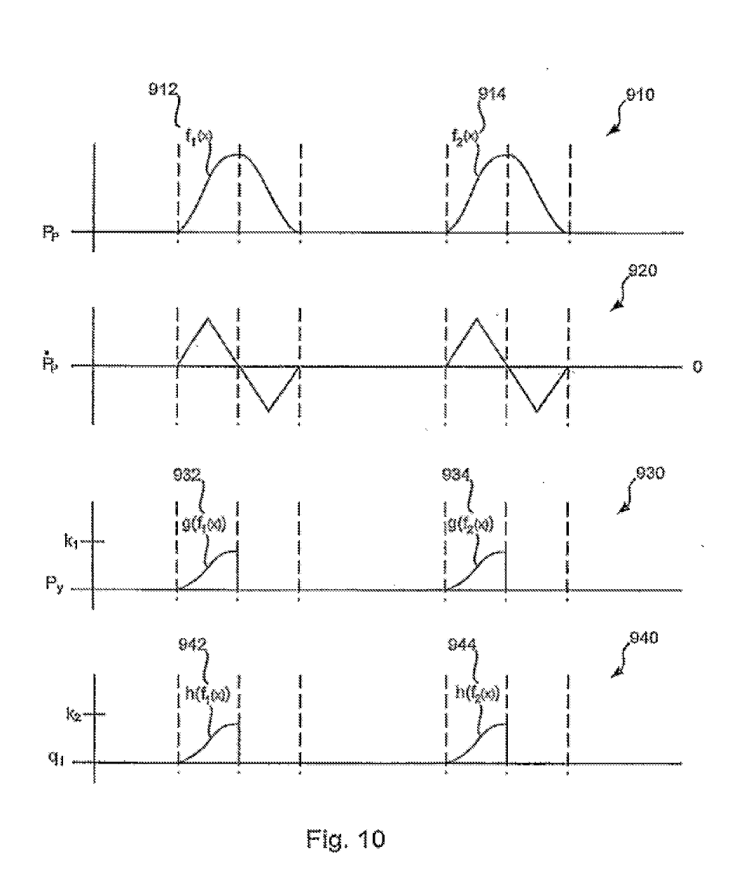

Turning to FIG. 10, four timing diagrams 910, 920, 930, 940 graphically depict providing ventilation in proportion to patient effort in accordance with one or more embodiments of the present invention. Timing diagram 910 depicts patient effort as a function of time, and timing diagram 920 depicts a derivative of patient effort as a function of time. As shown, when the derivative of patient effort is greater than zero (corresponding to an inspiration phase), patient effort is described as a function f(x). It should be noted that while timing diagram 910 shows patient effort as the same function repeating over time, that a first instance of f₁(x) 912 may differ substantially from the second instance of f₂(x) 914 depending upon the breathing pattern of the particular patient.

A timing diagram 930 depicts an effort by a ventilator to increase the pressure at the wye connection to offset a pressure decrease caused by patient effort. As shown, during the inspiration phase (i.e., when the derivative of patient effort is greater than zero), the ventilator attempts to raise the pressure at the wye connection as a function of patient effort, g(f₁(x)) 932. On a subsequent breath, the ventilator attempts to raise the pressure at the wye as a function of patient effort, g(f₂(x)) 934. In this particular case, the function g is a constant k₁, however, other time varying functions may be used in accordance with different embodiments of the present invention.

Similarly, during the inspiration phase, the ventilator increases the flow of gas to a patient as a function of patient effort, h(f₁(x)) 942. On a subsequent breath, the ventilator increases the flow of gas to a patient as a function of patient effort, h(f₂(x)) 944. In this particular case, the function h is a constant k₂, however, other time varying functions may be used in accordance with different embodiments of the present invention. In some cases, the functions g and h may be proportional or inversely proportional to patient effort. It should be noted that in the sense that gas delivery is provided as a function of patient effort, that patient effort may be determined based directly on patient effort (i.e., patient interpleural pressure), or on a first or higher order derivative of patient effort.

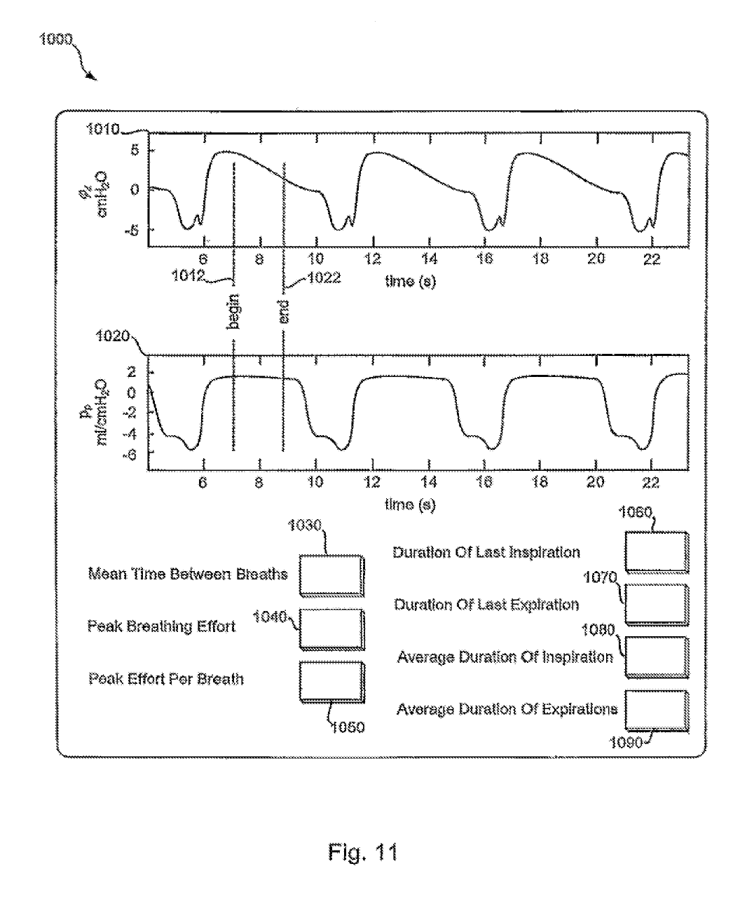

Turning to FIG. 11, an exemplary graphical interface 1000 showing the display of patient effort corresponding to an actively breathing patient in accordance with some embodiments of the present invention. Graphical interface 1000 includes a graphical display of filtered patient effort (φ_{d}) 1010, and patient effort (pₚ) 1020 each as a function of time. It should be noted that other indications of patient effort may be displayed in addition to those depicted or in place of those depicted depending upon the particular embodiment of the present invention.

In the depicted embodiment, time is displayed across a horizontal axis and the value of the respective patient effort value is displayed across a left axis. As time proceeds, the time increments across the horizontal axis are updated to reflect a window around the current time. In addition, two user movable vertical bars 1012, 1022 are disposed over graph 1010 and graph 1020. This allows a user to place a begin bar 1012 and an end bar 1022 at particular times to measure an event. The time difference between begin bar 1012 and end bar 1022 may be displayed to the user, along with the value of filtered patient effort and patient effort at the respective instants in time. In some cases, begin bar 1012 and end bar 1022 may be used via a keyboard command or a mouse command. Based on the disclosure provided herein one of ordinary skill in the art will recognize a variety of I/O that may be used to manipulate begin bar 1012 and end bar 1022 in relation to graphs 1010, 1020.

In addition, various metrics relating to the graphically displayed patient effort may be calculated and displayed via graphical interface 1000. For example, a mean time between breaths 1030 may be calculated and displayed. Such a mean time may be calculated based on a defined number of breaths, where a time between each of the breaths is calculated from the end of expiration to the beginning of subsequent inspiration. Based on the disclosure provided herein, one of ordinary skill in the art will appreciate a variety of approaches that may be used to calculate mean time between breaths in accordance with different embodiments of the present invention. As another example, a peak breathing effort 1040 may be displayed. Peak breathing effort 1040 may be the maximum value recorded on either of graph 1010 or graph 1020 over the course of a defined number of breaths depending upon the particular implementation. As yet another example, peak effort per breath 1050 may be displayed. Peak effort per breath 1050 may indicate the peak value of either graph 1010 or graph 1020 for a most current breath. Alternatively it may indicate the peak value of either graph 1010 or graph 1020 for a breath identified by begin bar 1012. As yet a further example, a duration of last inspiration 1060 may be displayed. Duration of the last inspiration 1060 indicates a time from when the onset of inspiration was detected until the end of inspiration was detected for the most recent breath. In one case, this may be achieved by detecting when a first derivative of the patient effort exceeds a threshold until it returns below the threshold. As another example, a duration of the last expiration 1070 may be displayed. In some cases, duration of the last expiration 1070 may be calculated by detecting when a first derivative of the patient effort falls below a threshold until the time when the first derivative returns above the threshold. As another example, an average duration of inspiration 1080 and an average duration of expiration 1090 may be displayed. The may be calculated by averaging a number of the previously discussed expiration durations and inspiration durations.

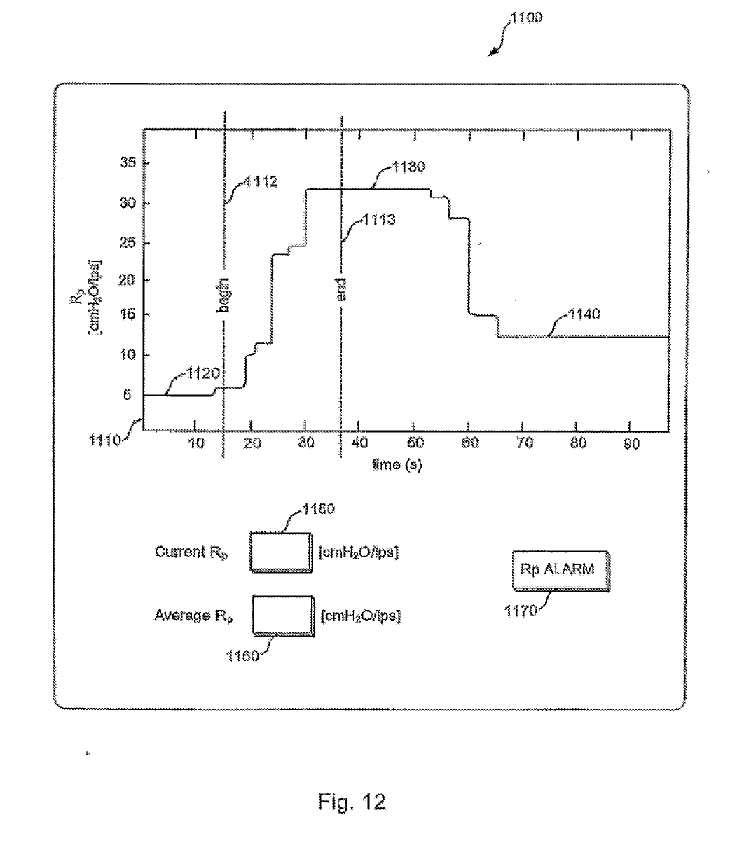

Turning to FIG. 12, an exemplary graphical interface 1100 showing the display of a respiratory parameter corresponding to an actively breathing patient in accordance with some embodiments of the present invention. In particular, a graph 1110 depicts an estimated value of the patient resistance parameter as a function of time. In some embodiments, the patient resistance parameter is referred to as “estimated Rp” because it is the result of calculation as distinguished from the actual value of the patient resistance. It should be noted that while graphical interface 1100 is described as showing estimated Rp, that other respiratory parameters may be displayed in accordance with different embodiments of the present invention. For example, graphical interface 1100 may be augmented to display lung compliance or leakage parameters, with these additionally displayed parameters determined using the same or similar set of equations as described herein. Based on the disclosure provided herein, one of ordinary skill in the art will recognize a variety of respiratory parameters that may be displayed. In some cases, the displayed respiratory parameters may be used by a monitoring clinician for real time assessment of a patient. Alternatively, or in addition, the displayed respiratory parameters may be used to determine a potential system malfunction or to indicate a disconnect of the patient from the ventilator. As one particular example, a dramatic increase in Rp may indicate a partial obstruction. Based on the disclosure provided herein, one of ordinary skill in the art will recognize a variety of advantages that may be achieved in accordance with one or more embodiments of the present invention.

As shown; time is displayed across a horizontal axis and the value of estimated Rp is displayed across a left axis. In some embodiments, as time proceeds, the time increments across the horizontal axis are updated to reflect a window around the current time. Additionally, in some embodiments, two user movable vertical bars 1112, 1113 are disposed over graph 1110. This allows a user to place a begin bar 1112 and an end bar 1113 at particular times to measure an event. The time difference between begin bar 1112 and end bar 1113 may be displayed to the user, along with the value of filtered patient effort and patient effort at the respective instants in time. In some cases, begin bar 1112 and end bar 1113 may be used via a keyboard command or a mouse command. Based on the disclosure provided herein one of ordinary skill in the art will recognize a variety of I/O that may be used to manipulate begin bar 1112 and end bar 1113 in relation to graph 1110.

In this particular example, for an initial period 1120 estimated Rp is initialized with a value of five (5) cmH₂O/Ips. At this time, the actual value of Rp is nearer to thirty (30) cmH₂O/Ips. Over a period of time, the algorithm used to determine the value of estimated Rp adaptively adjusts until the estimated value approximates the actual value for Rp during a period 1130. Sometime around the fifty (50) second mark, an obstruction is removed from the ventilation system resulting in a dramatic decrease in the actual value of Rp. At this point, the algorithm adaptively adjusts by lowering the value of estimated Rp until the estimated value approximates the actual value. During a period 1140, the value of estimated Rp remains approximately constant near the actual value of Rp.

In addition, various metrics relating to the graphically displayed resistance parameter may be calculated and displayed via graphical interface 1100. For example, a current Rp value 1150 may be displayed, and an average Rp value 1160 may be displayed. Average Rp value 1160 may be calculated by averaging a number of values for Rp over a particular time period. In addition, a visual alarm 1170 may be displayed. Such a visual alarm may be triggered whenever a predefined increase or decrease in the value of estimated Rp is detected. It should be noted that graphical interface 1100 may be augmented to display a variety of other information.

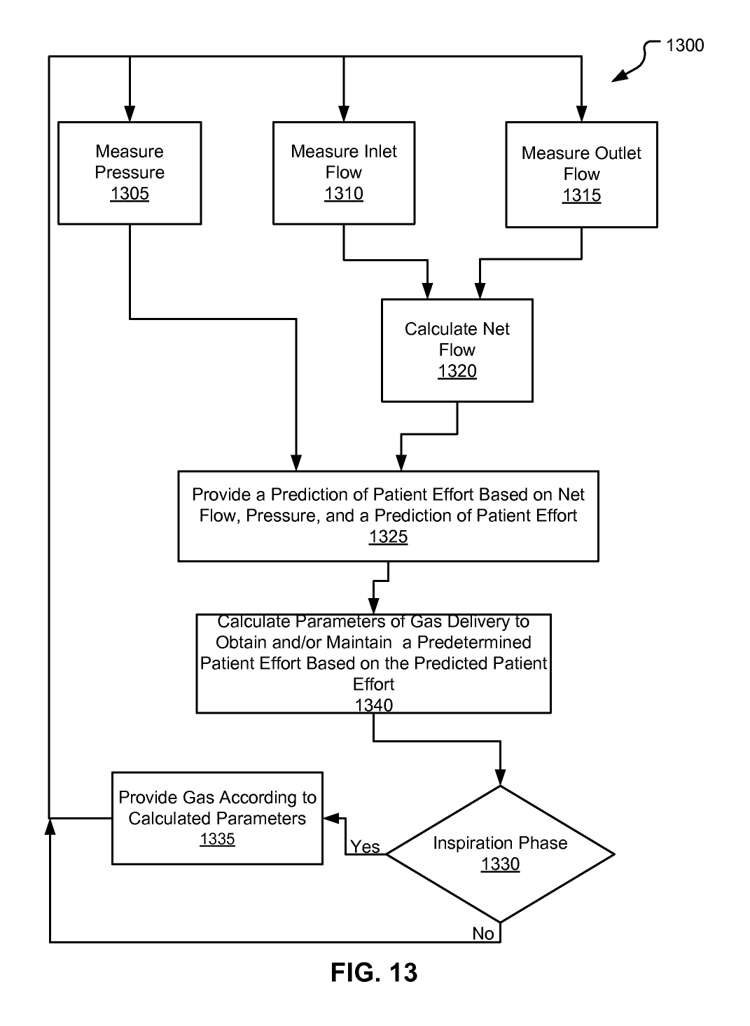

Turning to FIG. 13, a flow diagram 1300 illustrates a method for providing ventilation to obtain and/or maintain a predetermined patient effort in accordance with various embodiments of the present disclosure. Following flow diagram 1300, a pressure is measured (block 1305), an inlet flow is measured (block 1310), and an outlet flow is measured (block 1315). In some cases, the pressure is measured in a tube connecting a ventilator to a person being ventilated. In some cases, the pressure is measured near a gas inlet and/or near a gas outlet. In other cases, the pressure is measured near a junction of the gas inlet with the gas outlet. In various cases, the pressure measurement is a single point pressure measurement, while in other cases the pressure measurement is a multiple point pressure measurement and the measured pressure is a mathematical combination of two or more pressure measurements. Measuring the inlet flow may include measuring the flow of a single gas, or measuring the flows of two or more gases and aggregating the multiple flow values. Measuring the outlet flow may include, but is not limited to, measuring the flow of gas at the outlet of the ventilation system. The outlet flow is subtracted from the inlet flow at a particular instance to generate an instantaneous net flow (block 1320).

The net flow and measured pressure for a given instant are used to calculate an updated prediction of patient effort (block 1325). This process may be done using the approach discussed above in relation to FIG. 4. Desired gas delivery parameter(s) of gas to be delivered by the ventilator to obtain and/or maintain a predetermined patient effort are calculated based on the predicted patient effort (block 1340). In some embodiments, the predetermined patient effort is selected or input by an operator. In other embodiments, the predetermined patient effort is determined by the ventilator.

In additional embodiments of the present disclosure, the gas delivery parameters are flow and/or pressure. In this case, the pressure and flow of gas delivery necessary to obtain and/or maintain the predetermined patient effort are each calculated based on the predicted patient effort. For example, where predicted patient effort is determined to be a value at an instant x described by a function f(x) with a known pressure of g₁, then the predetermined patient effort may be described at an instant using the function f(y) at unknown pressure g₂. Accordingly, g₂ may be calculated using the following equation:

g₁(f(x))=g₂(f(y)).

Further in another example, where predicted patient effort is determined to be a value at an instant x described by a function f(x) with a known flow of h₁, then the predetermined patient effort may be described at an instant using the function f(y) at unknown flow h₂. Accordingly, the calculated flow at an instant may be calculated utilizing the following equation:

h₁(f(x))=h₂(f(y)).

Based on the disclosure provided herein, one of ordinary skill in the art will recognize other functions g and functions h that may be used in relation to different embodiments of the present disclosure to obtain the predetermined patient effort. The pressure used as a metric for delivering gas may be, but is not limited to, wye pressure or patient lung pressure. The flow used as a metric for delivering gas may be, but is not limited to, patient lung flow or inlet gas flow. In alternative embodiment, the predetermined patient effort may be calculated on a per breath or on a predetermined number breaths basis instead of based on a specific instant in time and/or a specific instant in time within a breath basis as shown in the equations above.

It is then determined whether the updated prediction of patient effort indicates an inspiration phase (block 1330). In some embodiments of the present disclosure, an inspiration phase is indicated where the derivative of patient effort \dot{p}_{P} is greater than zero. Where an inspiration phase is indicated (block 1330), gas is delivered to a recipient in accordance with the gas delivery parameters previously calculated (block 1335). Again, gas delivery parameters may include, but are not limited to, pressures and flows of gas or gas components (e.g., oxygen, air, nitrogen, helium, etc.) to be delivered to a patient. Otherwise, where an inspiration phase is not indicated (block 1330), gas delivery is not provided.

In an additional embodiment, any method for determining or estimating patient effort may be utilized to obtain and/or maintain a predetermined patient effort based on a calculated flow and/or pressure. This approach provides for gas delivery at a rate and/or pressure to provide and/or maintain the predetermined patient effort. Accordingly, this approach provides for increased patient comfort as well as less interference with a patient's own attempts at breathing.

The present invention provides novel systems, methods and devices delivering a gas in proportion to a patient effort. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A ventilation system, comprising:
a processor communicably coupled to a computer readable medium, wherein the computer readable medium includes instructions executable by the processor to:
receive a measured pressure value;
receive a net flow value;
calculate a patient effort value based on a relationship between patient effort, the measured pressure value and the net flow value;
update an interim value based at least in part on the patient effort value;
calculate a computed patient effort based at least in part on the interim value;
calculate a gas delivery metric to obtain a predetermined patient effort, wherein the gas delivery metric is based on the computed patient effort value; and
cause a gas to be delivered consistent with the gas delivery metric.

2. The system of claim 1, wherein the gas delivery metric is selected from: pressure, and flow.

3. The system of claim 2, wherein the flow is selected from: patient lung flow and inlet gas flow.

4. The system of claim 2, wherein the pressure is selected from: lung pressure and wye pressure.

5. The ventilation system of claim 1, wherein the computer readable medium further includes instructions executable by the processor to:
determine an inhalation phase based on the patient effort value.

6. The ventilation system of claim 5, wherein the inhalation phase is indicated when the calculated patient effort value is greater than zero,

7. The ventilation system of claim 5, wherein the system is operable to deliver the gas during the determined inhalation phase.

8. The ventilation system of claim 1, wherein the patient effort value is further calculated based on a combination of one or more intermediate values calculated using the measured pressure value and the net flow value, the one or more intermediate values selected from: an estimated normalized prediction error (ε), a filtered pressure value (z), a regression vector (φ^{T}), and a current estimated value of a parameter vector (θ).

9. The ventilation system of claim 1, wherein the relationship is a parameterized system input to output relationship.

10. The method of claim 9, wherein the parameterized system input to -output relationship is the regression form: z=θ^{T}φ+φ_{d}.

11. The method of claim 10, wherein the parameterized system input to output relationship is derived from a transfer function.

12. The ventilation system of claim 11, wherein the transfer function is derived from a model:
$$[ \begin{matrix}{\dot{p}}_{Y} \\ {\dot{p}}_{L}\end{matrix} ] = [ \begin{matrix}- \frac{1}{{C}_{T} {R}_{P}} & \frac{1}{{C}_{T} {R}_{P}} \\ \frac{1}{{C}_{L} {R}_{P}} & - \frac{1}{{C}_{L} {R}_{P}}\end{matrix} ] [ \begin{matrix}{p}_{Y} \\ {p}_{L}\end{matrix} ] + [ \begin{matrix}\frac{1}{{C}_{T}} & \frac{1}{{C}_{T}} & - \frac{1}{{C}_{T}} \\ 0 & 0 & 0\end{matrix} ] [ \begin{matrix}{q}_{AIR} \\ {q}_{O 2} \\ {q}_{E}\end{matrix} ] + [ \begin{matrix}0 & - \frac{1}{{C}_{T}} & 0 \\ 1 & 0 & - \frac{1}{{C}_{T}}\end{matrix} ] [ \begin{matrix}{\dot{p}}_{P} \\ {q}_{Tleak} \\ {q}_{Pleak}\end{matrix} ]$$

13. The ventilation system of claim 1, wherein the calculated gas delivery metric is utilized to maintain a predetermined patient effort during ventilation, wherein the gas delivery metric is based on the computed patient effort value.

14. A method for respiratory support, the method comprising:
measuring a pressure and providing a measured pressure;
measuring an inlet flow and an outlet flow, and providing a measured net flow;
using a relationship between a first value related to the measured pressure, a second value related to the measured net flow and a third value related to patient effort to calculate a prediction of patient effort from the measured pressure and measured net flow;
updating an interim value based at least in part on the prediction of patient effort;
calculating a computed patient effort based at least in part on the interim value; and
supplying a gas based on the computed patient effort to obtain a predetermined patient effort.

15. The method of claim 14, wherein supplying the gas based on the computed patient effort includes supplying the gas at a pressure to obtain the predetermined patient effort.

16. The method of claim 14, wherein supplying the gas based on the computed patient effort includes supplying the gas at a flow to obtain the predetermined patient effort,

17. The method of claim 14, wherein the prediction of patient effort is further based on a combination of one or more intermediate values derived from the measured pressure and measured net flow, the one or more intermediate values selected from: an estimated normalized prediction error (ε), a filtered pressure value (z), a regression vector (φ^{T}), and a current estimated value of a parameter vector (θ).

18. The method of claim 14, wherein the second value is selected from: a filtered version of the measured net flow, and the measured net flow. 19, The method of claim 14, wherein the first value is selected from: a filtered version of the measured pressure, and the measured pressure.

20. The method of claim 14, wherein the computed patient effort is a filtered patient effort signal.

21. The method of claim 14, wherein the computed patient effort is a linear function of a derivative of actual patient effort.

22. The method of claim 21, wherein the linear function includes a filter function.

23. The method of claim 14, wherein the relationship is a parameterized system input to output relationship.

24. The method of claim 23, wherein the parameterized system input to 3 output relationship is the regression form: z=θ^{T}φ+φ_{d}.

25. The method of claim 24, wherein the parameterized system input to output relationship is derived from a transfer function.

26. The method of claim 25, wherein the transfer function is derived from the model:
$$[ \begin{matrix}{\dot{p}}_{Y} \\ {\dot{p}}_{L}\end{matrix} ] = [ \begin{matrix}- \frac{1}{{C}_{T} {R}_{P}} & \frac{1}{{C}_{T} {R}_{P}} \\ - \frac{1}{{C}_{L} {R}_{P}} & \frac{1}{{C}_{L} {R}_{P}}\end{matrix} ] [ \begin{matrix}{p}_{Y} \\ {p}_{L}\end{matrix} ] + [ \begin{matrix}\frac{1}{{C}_{T}} & \frac{1}{{C}_{T}} & \frac{1}{{C}_{T}} \\ 0 & 0 & 0\end{matrix} ] [ \begin{matrix}{q}_{AIR} \\ {q}_{O 2} \\ {q}_{E}\end{matrix} ] + [ \begin{matrix}0 & - \frac{1}{{C}_{T}} & 0 \\ 1 & 0 & - \frac{1}{{C}_{T}}\end{matrix} ] [ \begin{matrix}{\dot{p}}_{p} \\ {q}_{Tleak} \\ {q}_{Pleak}\end{matrix} ]$$

27. The method of claim 14, wherein the calculated gas delivery metric is utilized to maintain a predetermined patient effort during ventilation, wherein the gas delivery metric is based on the computed patient effort value

28. A ventilation system, the system comprising:
a gas inlet;
a gas outlet;
a tube coupling the gas inlet and the gas outlet;
a pressure sensor, wherein the pressure sensor is operable to provide a measured pressure value indicating a pressure in the tube;
a first flow sensor, wherein the first flow sensor is operable to provide an inlet flow value indicating a flow associated with the gas inlet;
a second flow sensor, wherein the second flow sensor is operable to provide an outlet flow value indicating a flow associated with the gas outlet; and
a processor communicably coupled to a computer readable medium, wherein the computer readable medium includes instructions executable by the processor to:
receive a measured pressure value;
receive a net flow value;
calculate a patient effort value based on a relationship between patient effort, the measured pressure value and the net flow value;
update an interim value based at least in part on the patient effort value;
calculate a computed patient effort based at least in part on the interim value;
calculate a gas delivery metric based on the computed patient effort to obtain a predetermined patient effort;
determine an inhalation phase based on the computed patient effort; and
cause a gas to be delivered consistent with the gas delivery metric.

29. A ventilation system, the system comprising:
means for measuring a pressure and providing a measured pressure;
means for measuring an inlet flow and an outlet flow, and providing a measured net flow;
means for using a relationship between a first value related to the measured pressure, a second value related to the measured net flow and a third value related to patient effort to calculate a prediction of patient effort from the measured pressure and measured net flow;
means for updating an interim value based at least in part on the prediction of patient effort;
means for calculating a computed patient effort based at least in part on the interim value; and
means for supplying a gas based on the computed patient effort to maintain a predetermined patient effort.
